SEQUENCE LISTING (3 sequences)

SEQ ID NO: 1 - DNA, 924 bp, Artificial Sequence
  Human codon-optimized CLN1 open reading frame
atggcttctccggggtgtctgtggctgctggcagtggcactccttccctggacttgcgccagccgggctctgcagcacctcgaccctccagcccctcttccactggtgatttggcacggaatgggtgattcctgctgtaatcccctgtcaatgggagccatcaagaagatggtggagaagaagatccctggaatctacgtgctgtcactggagattggaaagaccctgatggaggacgtcgagaactccttcttcctcaatgtcaactctcaagtgaccaccgtctgccaggccctggccaaggacccgaagctgcagcaggggtataatgctatggggttcagccagggaggacagttccttcgggctgtggcccaacgctgccctagcccacccatgatcaacctgatctcagtgggtggccagcatcagggcgtgttcggacttccccggtgtcccggggaatcctctcatatctgcgacttcatccgcaaaactctcaatgcaggcgcttattcaaaggtcgtccaagagaggctggtgcaagccgagtactggcacgatcccattaaggaggacgtgtacagaaatcactcaatctttctggccgacattaaccaggagaggggaattaacgaatcatataagaagaatctcatggccctcaaaaagttcgtcatggtgaagttccttaacgatagcattgtggacccagtggacagcgaatggttcggattttaccgctcaggccaggcaaaagaaaccatccctctccaagagacttctctttacacccaagacagacttgggcttaaggaaatggataacgctggtcagctggtgttcctcgccaccgaaggtgaccatctgcagctcagcgaagagtggttctacgctcatatcatcccgtttcttggttgataa

SEQ ID NO: 2 - DNA, 921 bp, Homo sapiens
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 3 - protein, 306 aa, Homo sapiens
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

(19) United States
(12) Patent Application Publication
(10) Pub. No.: US 2022/0193268 A1
(43) Pub. Date: Jun. 23, 2022

(54) INTRATHECAL AND INTRAVENOUS COMBINATION GENE THERAPY FOR THE TREATMENT OF INFANTILE BATTEN DISEASE

(71) Applicants: The University of North Carolina at Chapel Hill, Chapel Hill, NC (US); Abeona Therapeutics, Inc., New York, NY (US)

(72) Inventors: Timothy J. MILLER, New York, NY (US); Steven J. GRAY, Southlake, TX (US)

(73) Assignee: The University of North Carolina at Chapel Hill, Chapel Hill, NC (US)

(21) Appl. No.: 17/607,315

(22) PCT Filed: Apr. 29, 2020
(86) PCT No.: PCT/US2020/030427

§ 371 Date: Oct. 28, 2021

Related U.S. Application Data
(60) Provisional application No. 62/840,360, filed on Apr. 29, 2019.

(51) Int. Cl. A61K 48/00; A61P 25/08; A61P 25/28; A61K 9/00; C12N 15/86

(52) U.S. Cl. CPC A61K 48/0075; A61P 25/08; A61P 25/28; A61K 9/0019; C12N 2750/14143; A61K 48/005; C12N 2750/14171; C12N 2750/14123; C12N 15/86

(57) ABSTRACT

Methods for treating IBD or an IBD related disorder in a subject in need thereof are provided that comprise combined intrathecal administration of a polynucleotide comprising a CLN1 open reading frame and intravenous administration of the polynucleotide. The polynucleotide comprising the CLN1 open reading frame is a wild-type CLN1 polynucleotide. In another aspect, the polynucleotide comprising the CLN1 open reading frame comprises codon-optimized polynucleotide sequence of the polynucleotide or its complement and is codon-optimized for expression in a human cell.

### CROSS REFERENCE TO RELATED APPLICATIONS

This application claims the benefit of priority to U.S. Provisional Application No. 62/840,360, filed on Apr. 29, 2019, the content of which is hereby incorporated by reference in its entirety for all purposes.

### INCORPORATION OF THE SEQUENCE LISTING

The contents of the text file submitted electronically herewith are incorporated herein by reference in their entirety: a computer readable format copy of the Sequence Listing (filename: ABEO_004_01WO_SeqList_ST25.TXT, date created: Apr. 27, 2020, file size: ˜5.6 kilobytes).

### BACKGROUND

Infantile neuronal lipofuscinosis (infantile Batten disease or “IBD”) or infantile neuronal ceroid lipofuscinosis (INCL) is caused by mutations in the CLN1 gene and is an autosomal recessive disorder. The CLN1 gene, located at 1p32, encodes a lysosomal enzyme called palmitoyl protein thioesterase 1 (PPT1). PPT1 deficient cells accumulate autoflurorescent storage material and become dysfunctional, leading to neuro-inflammation, neuron loss, and neurodegeneration. Some children with mutations in CLN1 have a later onset of symptoms and slower disease progression, which resembles juvenile onset disease and is more typically associated with mutations in the CLN3 gene. Prior treatments include enzyme replacement therapy, gene therapy and the administration of neural stem cells.

### SUMMARY OF THE DISCLOSURE

Provided herein are methods for treating IBD or an IBD related disorder in a subject in need thereof, comprising, or consisting essentially of, or yet further consisting of, intrathecal administration of a polynucleotide comprising a CLN1 open reading frame and subsequent intravenous administration of the polynucleotide, thereby treating IBD or an IBD related disorder. In one aspect, the polynucleotide comprising the CLN1 open reading frame comprises a wild-type CLN1 polynucleotide. In another aspect, the polynucleotide comprising the CLN1 open reading frame comprises codon-optimized polynucleotide sequence of the polynucleotide or its complement is codon-optimized for expression in a human cell. In one aspect, the polynucleotide comprises the nucleotide sequence of SEQ ID NO: 1 or SEQ ID NO: 2, or a nucleotide sequence having at least about 90% identical of each thereto or their complements, wherein the equivalent is identical at the codon-optimized nucleotides.

### BRIEF DESCRIPTION OF THE DRAWINGS

FIG. 1 shows a map of an illustrative CLN1 expression cassette.

FIG. 2 shows serum enzyme activity of PPT1 in mice administered scAAV9/CLN1 therapy. IT=lumbar intrathecal injection; IV=intravenous injection; Het=heterologous; KO=knock-out.

FIG. 3A shows survival curves for cohorts of CLN1 knockout mice intrathecally administered with the CLN1 AAV vector at 4 or 12 weeks of age. FIG. 3B shows survival curves for cohorts of CLN1 knockout mice intrathecally administered with the CLN1 AAV vector at 20 or 26 weeks of age.

FIG. 4A shows survival curves for cohorts of CLN1 knockout mice intrathecally or intrathecally+intravenously administered with the CLN1 AAV vector at 4 weeks of age. FIG. 4B shows survival curves for cohorts of CLN1 knockout mice intrathecally, intravenously, or intrathecally+intravenously administered with the CLN1 AAV vector at 20 weeks of age.

FIG. 5A shows survival curves for CLN1 knockout mice administered with the CLN1 AAV vector pre-symptom onset at different doses and via different administration routes. FIG. 5B shows survival curves for CLN1 knockout mice administered with the CLN1 AAV vector post-symptom onset at different doses and via different administration routes.

FIGS. 6A-6B shows swim speed assessment in the Morris Water Maze.

FIGS. 7A-7B show time to fall from inverted wire-hang.

FIG. 8 shows normalized physical capacity score (PSC) vs relative survival time for various mouse treatment groups.

FIG. 9A shows serum PPT1 levels in heterologous mice administered scAAV9/CLN1 therapy as neonates. FIG. 9B shows swimming speed in heterologous mice administered scAAV9/CLN1 therapy as neonates.

FIG. 10 shows PPT1 enzyme activities measured in different tissues of rats treated with scAAV9/CLN1 vector.

FIG. 11 shows levels of neutralizing antibody against AAV9 in rats treated with scAAV9/CLN1 vector.

FIG. 12 depicts a diagram of IBD symptom development in mice treated with scAAV9/CLN1 vector at different time points.

### DETAILED DESCRIPTION OF THE DISCLOSURE

### Definitions

Throughout this disclosure, various publications, patents and published patent specifications are referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R.I. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others. As used herein, the transitional phrase consisting essentially of (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of shall mean excluding more than trace elements of other ingredients and substantial method steps for administering the compositions disclosed herein. Aspects defined by each of these transition terms are within the scope of the present disclosure.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, or alternatively consists essentially of, or yet further consists of three major viral proteins: VP1, VP2 and VP3. In one embodiment, the AAV refers to of the serotype AAV1, AAV2, AAV4, AAVS, AAV6, AAV7, AAV8, AAV9, AAV10, AAV11, AAV12, AAV13, AAV PHP.B, or AAV rh74. The AAV may be a self-complementary AAV (scAAV).

The term “cell” as used herein may refer to either a prokaryotic or eukaryotic cell, optionally obtained from a subject or a commercially available source.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality. Non-limiting examples of equivalent polypeptides or polynucleotides include those having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide or polynucleotide sequence, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively at least 80%, or alternatively at least 85%, or alternatively at least 90%, or alternatively at least 95% identity, or at least 97% sequence identity to the reference polynucleotide, e.g., the wild-type polynucleotide. In embodiments relating to optimized polynucleotides or proteins, a biological equivalent has the desired percent identity while maintaining unchanged the nucleotide(s) or amino acid(s) of the reference nucleotide(s) or amino acid(s) (e.g., having at least 60%, or alternatively at least 65%, or alternatively at least 70%, or alternatively at least 75%, or alternatively at least 80%, or alternatively at least 85%, or alternatively at least 90%, or alternatively at least 95% identity thereto or for polypeptide or polynucleotide sequence), or a polypeptide which is encoded by a polynucleotide or its complement that hybridizes under conditions of high stringency to a polynucleotide encoding such polypeptide sequences.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

A “gene” refers to a polynucleotide containing at least one open reading frame (ORF) that is capable of encoding a particular polypeptide or protein after being transcribed and translated. A “gene product” or alternatively a “gene expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on it being operatively linked to an element which contributes to the initiation of, or promotes, transcription. “Operatively linked” intends the polynucleotides are arranged in a manner that allows them to function in a cell. In one aspect, this invention provides promoters operatively linked to the downstream sequences, e.g., suicide gene, VEGF, 165A VEGF, tet activator, etc.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, or alternatively consisting essentially of, or yet further consisting of purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, or an EF1 promoter. In some embodiments, the promoter is a chicken β-actin (“CBA”) promoter.

Additional non-limiting exemplary promoters with certain target specificity are provided herein below including but not limited to CMV, EFla, SV40, PGK1 (human or mouse), P5, Ubc, human beta actin, CAG, TRE, UAS, Ac5, Polyhedrin, CaMKIIa, Gall, TEF1, GDS, ADH1, CaMV35S, Ubi, H1, U6, and Alpha-1-antitrypsin. Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency. Non-limiting examples of enhancers include an RSV enhancer or a CMV enhancer.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise, or alternatively consist essentially of, or yet further consist of a protein's or peptide's sequence. As used herein the term “amino acid” refers to either natural and/or unnatural or synthetic amino acids, including glycine and both the D and L optical isomers, amino acid analogs and peptidomimetics.

As used herein, the term “vector” refers to a non-chromosomal nucleic acid comprising, or alternatively consisting essentially of, or yet further consisting of an intact replicon such that the vector may be replicated when placed within a cell, for example by a process of transformation. Vectors may be viral or non-viral. Viral vectors include retroviruses, adenoviruses, herpesvirus, bacculoviruses, modified bacculoviruses, papovirus, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. Exemplary non-viral vectors for delivering nucleic acid include naked DNA; DNA complexed with cationic lipids, alone or in combination with cationic polymers; anionic and cationic liposomes; DNA-protein complexes and particles comprising, or alternatively consisting essentially of, or yet further consisting of DNA condensed with cationic polymers such as heterogeneous polylysine, defined-length oligopeptides, and polyethylene imine, in some cases contained in liposomes; and the use of ternary complexes comprising, or alternatively consisting essentially of, or yet further consisting of a virus and polylysine-DNA. Examples of viral vectors include retroviral vectors.

In embodiments, the vector comprises an inducible promoter. In embodiments, the inducible promoter is an inducible tetracycline promoter. The Tet-Off and Tet-On Gene Expression Systems give researchers ready access to the regulated, high-level gene expression systems described by Gossen & Bujard (1992; Tet-Off) and Gossen et al. (1995; Tet-On). In the Tet-Off system, gene expression is turned on when tetracycline (Tc) or doxycycline (Dox; a Tc derivative) is removed from the culture medium. In contrast, expression is turned on in the Tet-On system by the addition of Dox. Both systems permit gene expression to be tightly regulated in response to varying concentrations of Tc or Dox. Maximal expression levels in Tet systems are very high and compare favorably with the maximal levels obtainable from strong, constitutive mammalian promoters such as CMV (Yin et al., 1996). Unlike other inducible mammalian expression systems, gene regulation in the Tet Systems is highly specific, so interpretation of results is not complicated by pleiotropic effects or nonspecific induction. In E. coli, the Tet repressor protein (TetR) negatively regulates the genes of the tetracycline-resistance operon on the Tn10 transposon. TetR blocks transcription of these genes by binding to the tet operator sequences (tetO) in the absence of Tc. TetR and tetO provide the basis of regulation and induction for use in mammalian experimental systems. In the Tet-On system, the regulatory protein is based on a “reverse” Tet repressor (rTetR) which was created by four amino acid changes in TetR (Hillen & Berens, 1994; Gossen et al., 1995). The resulting protein, rtTA (reverse tTA also referred to tetracycline activator protein), is encoded by the pTet-On regulator plasmid. This gene may be in a separate vector as the therapeutic gene or encoded on the same gene.

In a related embodiment, the vector further comprises, or alternatively consists essentially of, or yet further consists of a nucleic acid encoding a tetracycline activator protein; and a promoter that regulates expression of the tetracycline activator protein.

Other inducible systems useful in vectors, isolated cells, viral packaging systems, and methods described herein include regulation by ecdysone, by estrogen, progesterone, chemical inducers of dimerization, and isopropyl-beta-D1-thiogalactopyranoside (EPTG).

As used herein, the term “recombinant expression system” or “recombinant vector” refers to a genetic construct or constructs for the expression of certain genetic material formed by recombination.

A population of cells intends a collection of more than one cell that is identical (clonal) or non-identical in phenotype and/or genotype. A substantially homogenous population of cells is a population having at least 70%, or alternatively at least 75%, or alternatively at least 80%, or alternatively at least 85%, or alternatively at least 90%, or alternatively at least 95%, or alternatively at least 98% identical phenotype, as measured by pre-selected markers.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the term “codon-optimized” refers to a coding sequence that is optimized relative to a wild type coding sequence (e.g., a coding sequence for PPT1) to increase expression of the coding sequence by substituting one or more codons normally present in the coding sequence with a codon for the same (synonymous) amino acid. In some embodiments, the substitutions minimize rare codons (e.g., human codons), increase total GC content, decrease CpG content, remove cryptic splice donor or acceptor sites, and/or add or remove ribosomal entry sites, such as Kozak sequences. International PCT Publication No.: WO 2017/218450, published Dec. 21, 2017 (incorporated herein by reference) discloses codon-optimized CLN1 gene sequences, methods for producing and general methods for delivery of a monotherapy using the codon-optimized CLN1 gene.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, or alternatively consisting essentially of, or yet further consisting of the viral genome or part thereof, and a transgene. Adenoviruses (Ads) are a relatively well characterized, homogenous group of viruses, including over 50 serotypes. Ads do not require integration into the host cell genome. Recombinant Ad derived vectors, particularly those that reduce the potential for recombination and generation of wild-type virus, have also been constructed. Such vectors are commercially available from sources such as Takara Bio USA (Mountain View, Calif.), Vector Biolabs (Philadelphia, Pa.), and Creative Biogene (Shirley, N.Y.). Wild-type AAV has high infectivity and specificity integrating into the host cell's genome. See, Wold and Toth (2013) Curr. Gene. Ther. 13(6):421-433, Hermonat & Muzyczka (1984) Proc. Natl. Acad. Sci. USA 81:6466-6470, and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise, or alternatively consist essentially of, or yet further consist of a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the term “signal peptide” or “signal polypeptide” intends an amino acid sequence usually present at the N-terminal end of newly synthesized secretory or membrane polypeptides or proteins. It acts to direct the polypeptide to a specific cellular location, e.g. across a cell membrane, into a cell membrane, or into the nucleus. In some embodiments, the signal peptide is removed following localization. Examples of signal peptides are well known in the art. Non-limiting examples are those described in U.S. Pat. Nos. 8,853,381, 5,958,736, and 8,795,965.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsidated” means enclosed within a viral capsid.

As used herein, the term “helper” in reference to a virus or plasmid refers to a virus or plasmid used to provide the additional components necessary for replication and packaging of a viral particle or recombinant viral particle, such as the modified AAV disclosed herein. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross - hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising, consisting essentially of, or consisting of one or more heterologous nucleic acid (HNA) sequences and one or more AAV inverted terminal repeat sequences (ITRs). Such AAV vectors can be replicated and packaged into infectious viral particles when present in a host cell that provides the functionality of rep and cap gene products; for example, by transfection of the host cell. In embodiments, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vector may also contain elements for manufacturing purposes, e.g., antibiotic resistance genes, etc., but these are not encapsidated and thus do not form part of the AAV particle.

An “AAV virion” or “AAV viral particle” or “AAV viral vector” or “AAV vector particle” or “AAV particle” refers to a viral particle composed of at least one AAV capsid protein and an encapsidated polynucleotide AAV vector. Thus, production of AAV vector particle necessarily includes production of AAV vector, as such a vector is contained within an AAV vector particle.

In some embodiments, the AAV is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. U.S. Pat. No. 9,434,928 also provide the sequences of the capsid proteins and a self-complementary genome. In one aspect, the genome is a self-complementary genome. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921- 13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV- 10, AAV-11, AAV- 12, AAV-13, AAV PHP.B and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art. Other suitable AAV particles are described in PCT/US2019/064396 filed Dec. 4, 2019, which is hereby incorporated by reference in its entirety, but with particularity with respect to the AAV particles and AAV capsid proteins.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises, or alternatively consists essentially of, or yet further consists of a response whose existence merely is confirmed, whereas a response that is quantified generally comprises, or alternatively consists essentially of, or yet further consists of a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises, or alternatively consists essentially of, or yet further consists of a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

A “composition” is intended to mean a combination of active polypeptide, polynucleotide or antibody and another compound or composition, inert (e.g., a detectable label) or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

A “subject” of diagnosis or treatment is a cell or an animal such as a mammal, or a human. A subject is not limited to a specific species and includes non-human animals subject to diagnosis or treatment and are those subject to infections or animal models, for example, simians, murines, such as, rats, mice, chinchilla, canine, such as dogs, leporids, such as rabbits, livestock, sport animals, and pets. Human patients are included within the term as well.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise, or alternatively consist essentially of, or yet further consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof

The term “IBD related disorder” as used herein refers to a disease, disorder, syndrome, or condition that is caused by or a symptom of decreased or altered expression of the CLN1 gene in a subject relative to the expression level or activity in a normal subject, a subject not exhibiting symptoms, or in a population.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, in some embodiments the effective amount is the amount sufficient to result in regaining part or full function of a gene that is deficient in a subject. In other embodiments, the effective amount of an AAV viral particle is the amount sufficient to result in expression of a gene in a subject. The skilled artisan will be able to determine appropriate amounts depending on these and other factors.

The term “vg” refers to viral units provided for gene therapy and are typically noted as vg/kg of the subject. The experiments described herein are amounts that were used to treat mice, and it is inferred that when the subject being treated is not a mouse, the amount is converted to an amount that is appropriate for the subject being treated, e.g., a human, an infant or newborn. In one aspect, the amount administered is in vg/kg and therefore accounts for difference in size and body weight of the subject being treated.

In embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

As used herein, the term “modified,” as applied to a polynucleotide or polypeptide sequence, refers to a sequence that differs from a wild-type sequence due to one or more deletions, additions, substitutions, or any combination thereof.

### Modes for Carrying Out the Disclosure

Provided herein are methods for treating IBD or an IBD related disorder in a subject in need thereof, comprising, or consisting essentially of, or yet further consisting of, intrathecal administration of a polynucleotide comprising a CLN1 open reading frame and subsequent intravenous administration of the polynucleotide, thereby treating IBD or an IBD related disorder. Optionally, the intravenous administration may precede the intrathecal administration.

In one aspect, the polynucleotide comprising the CLN1 open reading frame is part of a vector genome, and delivery of the polynucleotide is achieved by administering a viral particle comprising the vector genome. Therefore, in one aspect, provided herein are methods for treating infantile Batten disease (IBD) or an IBD related disorder in a subject comprising combined intrathecal and intravenous administration of effective amounts of viral particles comprising the polynucleotide.

Also provided are AAV vector particles, AAV vectors, and capsid proteins that find use in delivering the polynucleotide. The viral particle may be an AAV viral particle; for example, an AAV9 viral particle.

In one aspect, the polynucleotide comprising the CLN1 open reading frame comprises a wild-type CLN1 polynucleotide. In another aspect, the polynucleotide comprising the CLN1 open reading frame comprises a codon-optimized polynucleotide sequence of CLN1 or its complement is codon-optimized for expression in a human cell. In one aspect, the polynucleotide comprises the nucleotide sequence of SEQ ID NO: 1 or SEQ ID NO: 2, or a nucleotide sequence having at least about 80%, at least about 85%, at least about 90%, at least about 95%, at least about 98%, or at least about 99% identity of each thereto or their complements, wherein the equivalent is identical at the codon-optimized nucleotides. In one aspect, the polynucleotide comprises the nucleotide sequence encoding a polypeptide sequence having at least about 80%, at least about 85%, at least about 90%, at least about 95%, at least about 98%, at least about 99%, or 100% identity to SEQ ID NO: 3.

SEQ ID NO: 1: Human codon-optimized CLN1 open
reading frame (added stop codon is underlined)
ATGGCTTCTCCGGGGTGTCTGTGGCTGCTGGCAGTGGCACTCCTTCCCTG
GACTTGCGCCAGCCGGGCTCTGCAGCACCTCGACCCTCCAGCCCCTCTTC
CACTGGTGATTTGGCACGGAATGGGTGATTCCTGCTGTAATCCCCTGTCA
ATGGGAGCCATCAAGAAGATGGTGGAGAAGAAGATCCCTGGAATCTACGT
GCTGTCACTGGAGATTGGAAAGACCCTGATGGAGGACGTCGAGAACTCCT
TCTTCCTCAATGTCAACTCTCAAGTGACCACCGTCTGCCAGGCCCTGGCC
AAGGACCCGAAGCTGCAGCAGGGGTATAATGCTATGGGGTTCAGCCAGGG
AGGACAGTTCCTTCGGGCTGTGGCCCAACGCTGCCCTAGCCCACCCATGA
TCAACCTGATCTCAGTGGGTGGCCAGCATCAGGGCGTGTTCGGACTTCCC
CGGTGTCCCGGGGAATCCTCTCATATCTGCGACTTCATCCGCAAAACTCT
CAATGCAGGCGCTTATTCAAAGGTCGTCCAAGAGAGGCTGGTGCAAGCCG
AGTACTGGCACGATCCCATTAAGGAGGACGTGTACAGAAATCACTCAATC
TTTCTGGCCGACATTAACCAGGAGAGGGGAATTAACGAATCATATAAGAA
GAATCTCATGGCCCTCAAAAAGTTCGTCATGGTGAAGTTCCTTAACGATA
GCATTGTGGACCCAGTGGACAGCGAATGGTTCGGATTTTACCGCTCAGGC
CAGGCAAAAGAAACCATCCCTCTCCAAGAGACTTCTCTTTACACCCAAGA
CAGACTTGGGCTTAAGGAAATGGATAACGCTGGTCAGCTGGTGTTCCTCG
CCACCGAAGGTGACCATCTGCAGCTCAGCGAAGAGTGGTTCTACGCTCAT
ATCATCCCGTTTCTTGGTTGATAA
SEQ ID NO: 2 (Wild-type CLN1)-is known in the art,
e.g., as disclosed by provided by genenames.org/
data/gene-symbol-report/#!/hgnc_id/HGNC:9325
(last accessed on Apr. 29, 2019), which is shown
below:
ATGGCGTCGCCCGGCTGCCTGTGGCTCTTGGCTGTGGCTCTCCTGCCATG
GACCTGCGCTTCTCGGGCGCTGCAGCATCTGGACCCGCCGGCGCCGCTGC
CGTTGGTGATCTGGCATGGGATGGGAGACAGCTGTTGCAATCCCTTAAGC
ATGGGTGCTATTAAAAAAATGGTGGAGAAGAAAATACCTGGAATTTACGT
CTTATCTTTAGAGATTGGGAAGACCCTGATGGAGGACGTGGAGAACAGCT
TCTTCTTGAATGTCAATTCCCAAGTAACAACAGTGTGTCAGGCACTTGCT
AAGGATCCTAAATTGCAGCAAGGCTACAATGCTATGGGATTCTCCCAGGG
AGGCCAATTTCTGAGGGCAGTGGCTCAGAGATGCCCTTCACCTCCCATGA
TCAATCTGATCTCGGTTGGGGGACAACATCAAGGTGTTTTTGGACTCCCT
CGATGCCCAGGAGAGAGCTCTCACATCTGTGACTTCATCCGAAAAACACT
GAATGCTGGGGCGTACTCCAAAGTTGTTCAGGAACGCCTCGTGCAAGCCG
AATACTGGCATGACCCCATAAAGGAGGATGTGTATCGCAACCACAGCATC
TTCTTGGCAGATATAAATCAGGAGCGGGGTATCAATGAGTCCTACAAGAA
AAACCTGATGGCCCTGAAGAAGTTTGTGATGGTGAAATTCCTCAATGATT
CCATTGTGGACCCTGTAGATTCGGAGTGGTTTGGATTTTACAGAAGTGGC
CAAGCCAAGGAAACCATTCCCTTACAGGAGACCTCCCTGTACACACAGGA
CCGCCTGGGGCTAAAGGAAATGGACAATGCAGGACAGCTAGTGTTTCTGG
CTACAGAAGGGGACCATCTTCAGTTGTCTGAAGAATGGTTTTATGCCCAC
ATCATACCATTCCTTGGATGA

Suitable CLN1 genes encode a PPT1 protein having at least about 70%, at least about 75%, at least about 80%, at least about 85%, at least about 90%, at least about 95% identity, at least about 99% identity or 100% identity to SEQ ID NO: 3: MASPGCLWLLAVALLPWTCASRALQHLDPPAPLPLVIWHGMGD SCCNPLSMGAIKKM VEKKIPGIYVLSLEIGKTLMEDVENSFFLNVNSQVTTVCQALAKDPKLQQGYNAMGFSQ GGQFLRAVAQRCPSPPMINLISVGGQHQGVFGLPRCPGESSHICDFIRKTLNAGAYSKVV QERLVQAEYWHDPIKEDVYRNHSIFLADINQERGINESYKKNLMALKKFVMVKFLND SI VDPVD SEWFGFYRSGQAKETIPLQETSLYTQDRLGLKEMDNAGQLVFLATEGDHLQLS EEWFYAHIIPFLG. The PPT1 protein may have up to 1, 2, 3, 4, 5, 6, 7, 8, 9, or 10 amino acids different from SEQ ID NO: 3. The PPT1 protein may have up to 1, 2, 3, 4, 5, 6, 7, 8, 9, or 10 amino acids deleted from SEQ ID NO: 3.

The subject may be a mammal; for example, a human. The human may be an infant human; for example, from about 2 months to about 24 months, about 2 years to 12 years old, about 2 years to 5 years old, or 2 years to 5 years old.

The polynucleotide can be operably linked to additional elements, e.g., wherein the polynucleotide is operably linked to a promoter; and/or wherein the promoter is a chicken beta actin promoter; and/or wherein the polynucleotide is operably linked to an enhancer; and/or wherein the enhancer is a cytomegalovirus enhancer; and/or wherein the polynucleotide is operably linked to an intron; and/or wherein the intron is a hybrid/modified MVM intron; and/or wherein the polynucleotide is operably linked to a polyadenylation signal; and/or wherein the polyadenylation signal is a bovine growth hormone polyadenylation signal.

The polynucleotide may be part of a vector genome for delivery of the polynucleotide, and optionally the vector genome comprising the polynucleotide is packaged into viral particles comprising one or more of wild-type capsid proteins, mutated capsid proteins, tissue tropic capsid protein, and a modified capsid protein with altered tropism compared to a wild-type capsid protein, and wherein the modified capsid protein is optionally liver-detargeted.

The vector may comprise at least one adeno-associated virus (AAV) inverted terminal repeat (ITR), e.g., the vector comprises two AAV ITRs; and/or wherein the two AAV ITRs have the same nucleotide sequence; and/or wherein the two AAV ITRs have different nucleotide sequences; and/or wherein the AAV ITRs are AAV2 ITRs; and/or wherein the viral vector is self-complementary AAV genome.

In one aspect, the vector genome comprising the polynucleotide comprises an enhancer, a promoter, an intron, a human CLN1 open reading frame, and a polyadenylation site. In a further aspect, the polynucleotide comprises an AAV ITR, an enhancer, a promoter, an intron, a human CLN1 open reading frame, a polyadenylation site, and an AAV ITR. In a yet further aspect, the polynucleotide comprises a CMV enhancer, a chicken beta actin promoter, a hybrid/modified MVM intron, a human CLN1 open reading frame, and a bovine growth hormone polyadenylation site. In a yet further aspect, the polynucleotide comprises a mutant AAV ITR, a CMV enhancer, a chicken beta actin promoter, a hybrid/modified MVM intron, a human CLN1 open reading frame, a bovine growth hormone polyadenylation site, and a wild-type AAV ITR. In a yet further aspect, the polynucleotide comprises SEQ ID NO: 1 or 2, or an equivalent of each thereof.

The polynucleotide or the viral particle comprising the polynucleotide is administered in an amount to express functional CLN1 (i.e., a PPT1 protein) in the subject. The amount administered can be for transient or for extended expression of the functional CLN1 (i.e., a PPT1 protein) in the subject.

In one aspect, the amount administered intrathecally is the same as or different than the amount delivered intravenously.

In a further aspect, the polynucleotide or the viral particle comprising the polynucleotide is administered intrathecally and intravenously to the subject pre-symptom or post-symptom onset. Children with IBD generally develop symptoms between 2 and 24 months; often by about 18 months of age.

The amount to be delivered will vary with the subject and the disease being treated. In one aspect, the amount is considered median or high dose. For the purpose of illustration only, the amounts to be delivered (using a mouse as an example) is from about 2.0×10¹¹ vg to about 8.0×10¹¹ vg or 2.0×10¹¹ vg/kg to about 8.0×10¹¹ vg/kg. In a further aspect, the polynucleotide is delivered in an amount from about 7.0×10¹⁰ vg to about 8.0×10¹¹ vg or from 7.0×10¹° vg/kg to about 8.0×10¹¹ vg/kg. In a yet further aspect, the administration is delivered pre-symptom onset and in an amount from about 7.0×10¹⁰ vg (or vg/kg) to about 8.0×10¹¹ vg (or vg/kg). In a yet further aspect, the polynucleotide is administered in an amount from about 7.0×10¹¹ vg (or vg/kg) to about 8.0×10¹¹ vg (or vg/kg).

In one aspect, the amount of viral particles for intrathecal administration in human is from about 1.0×10¹² vg to about 1.0×10¹⁷ vg. In embodiments, the amount for intrathecal administration in human is about 1.0×10¹² vg to about 1.0×10¹⁴ vg, about 1.0×10¹³ vg to about 1.0×10¹⁵ vg, about 1.0×10¹⁴ vg to about 1.0×10¹⁶ vg, about 1.0×10¹⁵ vg to about 1.0×10¹⁷ vg, about 1.0×10¹³ vg to about 1.0×10¹⁴ vg, about 1.0×10¹⁴ vg to about 1.0×10¹⁵ vg, or about 1.0×10¹⁵ vg to about 1.0×10¹⁶ vg.

In one aspect, the amount of viral particles for intravenous administration in human is about 1.0×10¹¹ vg/kg to about 2.0×10¹⁶ vg/kg. In embodiments, the amount for intravenous administration in human is about 1.0×10¹¹ vg/kg to about 2.0×10¹³ vg/kg, about 1.0×10¹² vg/kg to about 2.0×10¹⁴ vg/kg, about 1.0×10¹³ vg/kg to about 2.0×10¹⁵ vg/kg, about 1.0×10¹⁴ vg/kg to about 2.0×10¹⁶ vg/kg, about 1.0×10¹² vg/kg to about 2.0×10¹³ vg/kg, about 1.0×10¹³ vg/kg to about 2.0×10¹⁴ vg/kg, about 1.0×10¹⁴ vg/kg to about 2.0×10¹⁵ vg/kg.

In addition to the treatment of IBD, the methods may be used to treat any disorder associated with expression of the CLN1 gene such as infantile, late-infantile, juvenile, or adult-onset neuronal ceroid lipofuscinosis. They can be used for other IBD related disorders that result from low or aberrant expression of CLN1, some of which are described in WO 2017/218450, incorporated herein by reference.

Also provided is a kit comprising a pharmaceutical composition comprising the CLN1 polynucleotide in a pharmaceutically acceptable carrier and instructions for use in the methods as disclosed herein.

### Methods of Producing AAV Viral Particles

A variety of approaches may be used to produce AAV viral vectors. In embodiments, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In embodiments, the cell is a packaging or helper cell line. In embodiments In aspects, the helper cell line is eukaryotic cell; for example, an HEK 293 cell or 293T cell. In embodiments, the helper cell is a yeast cell or an insect cell.

In embodiments, the cell comprises a nucleic acid encoding a tetracycline activator protein; and a promoter that regulates expression of the tetracycline activator protein. In embodiments, the promoter that regulates expression of the tetracycline activator protein is a constitutive promoter. In embodiments, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, see, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing AAV particles comprising transfecting a packaging cell line with any one of the AAV helper plasmids disclosed herein; and any one of the AAV vectors disclosed herein. In embodiments, the AAV helper plasmid and AAV vector are co-transfected into the packaging cell line. In embodiments, the cell line is a mammalian cell line, for example, human embryonic kidney (HEK) 293 cell line. The disclosure provides cells comprising any one of the AAV vectors and/or AAV particles disclosed herein.

### Pharmaceutical Compositions

The disclosure provides pharmaceutical compositions comprising any one of the AAV vectors, AAV capsids and/or AAV particles described herein. Typically, the AAV particles are administered for therapy.

The pharmaceutical composition, as described herein, may be formulated by any methods known or developed in the art of pharmacology, which include but are not limited to contacting the active ingredients (e.g., viral particles or recombinant vectors) with an excipient or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In embodiments, the pharmaceutical composition is formulated as a nanoparticle. In embodiments, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one -half or one-third of such a dosage. The formulations of the invention can include one or more excipients, each in an amount that together increases the stability of the viral vector, increases cell transfection or transduction by the viral vector, increases the expression of viral vector encoded protein, and/or alters the release profile of viral vector encoded proteins. In embodiments, the pharmaceutical composition comprises an excipient. Non limiting examples of excipients include solvents, dispersion media, diluents, or other liquid vehicles, dispersion or suspension aids, surface active agents, isotonic agents, thickening or emulsifying agents, preservatives, or combination thereof

In embodiments, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

### FURTHER NUMBERED EMBODIMENTS

Further numbered embodiments of the present disclosure are provided as follows:

Embodiment 1. A method for treating infantile Batten disease (IBD) or an IBD related disorder in a subject in need thereof, comprising intrathecal administration of a first polynucleotide comprising a CLN1 open reading frame and intravenous administration of a second polynucleotide comprising a CLN1 open reading frame, thereby treating IBD or an IBD related disorder.

Embodiment 2. The method of Embodiment 1, wherein the intrathecal administration precedes the intravenous administration.

Embodiment 3. The method of Embodiment 1 or 2, wherein the CLN1 open reading frame comprises a wild-type CLN1 polynucleotide, a codon-optimized sequence, or a nucleotide sequence having at least about 90% identity of each thereto.

Embodiment 4. The method of Embodiment 3, wherein the codon-optimized sequence is SEQ ID NO: 1.

Embodiment 5. The method of any one of Embodiments 1-4, wherein the CLN1 open reading frame encodes a polypeptide sequence having at least about 90% identity to SEQ ID NO: 3.

Embodiment 6. The method of any one of Embodiments 1-5, wherein the subject is a human patient.

Embodiment 7. The method of any one of Embodiments 1-6, wherein the CLN1 open reading frame is operably linked to a promoter.

Embodiment 8. The method of Embodiment 7, wherein the promoter is a chicken beta actin promoter.

Embodiment 9. The method of any one of Embodiments 1-8, wherein the CLN1 open reading frame is operably linked to an enhancer.

Embodiment 10. The method of Embodiment 9, wherein the enhancer is a cytomegalovirus enhancer.

Embodiment 11. The method of any one of Embodiments 1-10, wherein the CLN1 open reading frame is operably linked to an intron.

Embodiment 12. The method of Embodiment 11, wherein the intron is a hybrid/modified MVM intron.

Embodiment 13. The method of any one of Embodiments 1-12, wherein the CLN1 open reading frame is operably linked to a polyadenylation signal.

Embodiment 14. The method of Embodiment 13, wherein the polyadenylation signal is a bovine growth hormone polyadenylation signal.

Embodiment 15. The method of any one of Embodiments 1-14, wherein administration of the polynucleotide comprises administering a vector comprising the polynucleotide.

Embodiment 16. The method of Embodiment 15, wherein the vector is packaged into viral particles comprising one or more of wild-type capsid proteins, mutated capsid proteins, tissue tropic capsid proteins, or modified capsid proteins, wherein the modified capsid protein has altered tropism compared to a wild-type capsid protein.

Embodiment 17. The method of Embodiment 15, wherein the vector further comprises at least one adeno-associated virus (AAV) inverted terminal repeat (ITR).

Embodiment 18. The method of Embodiment 17, wherein the vector comprises two AAV ITRs.

Embodiment 19. The method of Embodiment 18, wherein the two AAV ITRs have the same nucleotide sequence.

Embodiment 20. The method of Embodiment 18, wherein the two AAV ITRs have different nucleotide sequences.

Embodiment 21. The method of Embodiment 19 and 20, wherein the AAV ITRs are AAV2 ITRs.

Embodiment 22. The method of any one of Embodiments 16-21, wherein the viral vector is self-complementary AAV genome.

Embodiment 23. The method of any one of Embodiments 15-22, wherein the vector comprises a polynucleotide comprising an enhancer, a promoter, an intron, a human CLN1 open reading frame, and a polyadenylation site.

Embodiment 24. The method of any one of Embodiments 15-22, wherein the vector comprises a polynucleotide comprising an AAV ITR, an enhancer, a promoter, an intron, a human CLN1 open reading frame, a polyadenylation site, and an AAV ITR.

Embodiment 25. The method of any one of Embodiment 15-22, wherein the vector comprises a polynucleotide comprising a CMV enhancer, a chicken beta actin promoter, a hybrid/modified MVM intron, a human CLN1 open reading frame, and a bovine growth hormone polyadenylation site.

Embodiment 26. The method of any one of Embodiments 15-22, wherein the vector comprises a polynucleotide comprising a mutant AAV ITR, a CMV enhancer, a chicken beta actin promoter, a hybrid/modified MVM intron, a human CLN1 open reading frame, a bovine growth hormone polyadenylation site, and a wild-type AAV ITR.

Embodiment 27. The method of any one of Embodiments 1-26, wherein the polynucleotide is administered in an amount to express functional CLN1 in the subject.

Embodiment 28. The method of Embodiment 27, wherein the polynucleotide is administered in an amount for an extended expression of the functional CLN1 in the subject.

Embodiment 29. The method of any one of Embodiments 1-28, wherein the amount administered intrathecally is the same or different than the amount delivered intravenously.

Embodiment 30. The method of any of Embodiments 1-29, wherein the polynucleotide is administered intrathecally to the subject pre-symptom.

Embodiment 31. The method of any one of Embodiments 15-30, wherein the amount of vector for intrathecal administration is from about 1.0×10¹⁴ vg to about 1.0×10¹⁵ vg.

Embodiment 32. The method of any one of Embodiments 15-31, wherein the amount of vector for intravenous administration is from about 1.0×10¹³ vg/kg to about 2.0×10¹⁴ vg/kg.

Embodiment 33. The method of any one of Embodiments 1-32, wherein the intrathecal administration and the intravenous administration are performed post-symptom onset.

Embodiment 34. The method of any one of Embodiments 1-32, wherein the intrathecal administration and the intravenous administration are performed pre-symptom onset.

Embodiment 35. The method of any one of Embodiments 1-34, wherein the disorder is infantile, late-infantile, juvenile, or adult-onset neuronal ceroid lipofuscinosis.

Embodiment 36. The method of any one of Embodiments 15-35, wherein the vector is an AAV vector.

Embodiment 37. The method of Embodiment 37, wherein the AAV vector is an AAV9 vector.

Embodiment 38. The method of Embodiment 36 or 37, wherein the AAV vector is encapsidated in a wild-type capsid protein.

Embodiment 39. The vector of Embodiment 36 or 37, wherein the AAV vector is encapsidated in a modified capsid protein with altered tropism compared to a wild-type capsid protein.

Embodiment 40. The method of Embodiment 49, wherein the modified capsid protein is liver-detargeted.

Embodiment 41. A kit comprising a pharmaceutical composition comprising the CLN polynucleotide in a pharmaceutically acceptable carrier and instructions for use in the methods of any one of Embodiments 1-40.

### EXAMPLES

### Example 1

### Intrathecal and Intravenous Administration of AAV Expressing PPT1 Protein

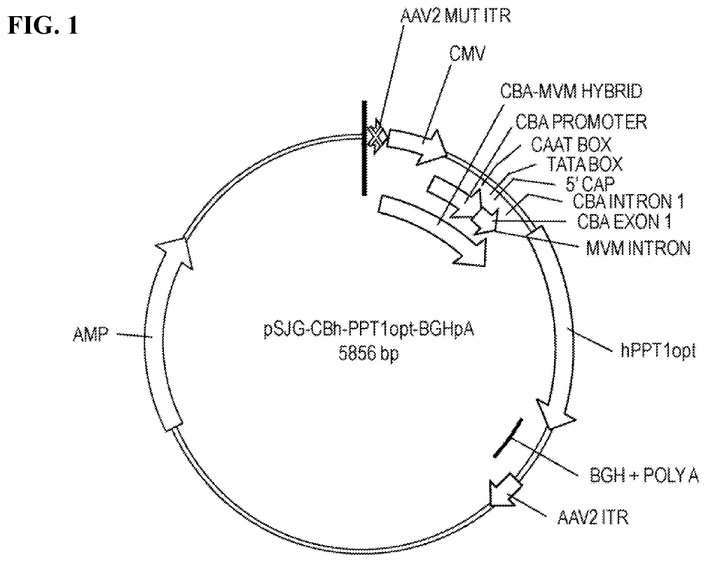

An AAV vector genome cassette was developed to express a PPT1 protein encoded by a CLN1 ORF. This cassette was designed to provide maximal expression from a self-complementary AAV genome that would be packaged within multiple AAV capsids. The cassette comprises, in 5′ to 3′ orientation: mutant AAV2 ITR, CMV enhancer, chicken beta actin promoter, hybrid/modified MVM intron, codon optimized human CLN1 ORF, bovine growth hormone polyadenylation site, and wild-type (WT) AAV2 ITR (FIG. 1). The expression of CLN1 was verified by transfecting the expression cassette into HEK293 cells and the expressed protein was detected in the cells and media by Western blot.

The CLN1 expression cassette was packaged within a wild-type AAV9 capsid and the resulting AAV viral particle was used to dose CLN1 knockout mice intrathecally and/or intravenously.

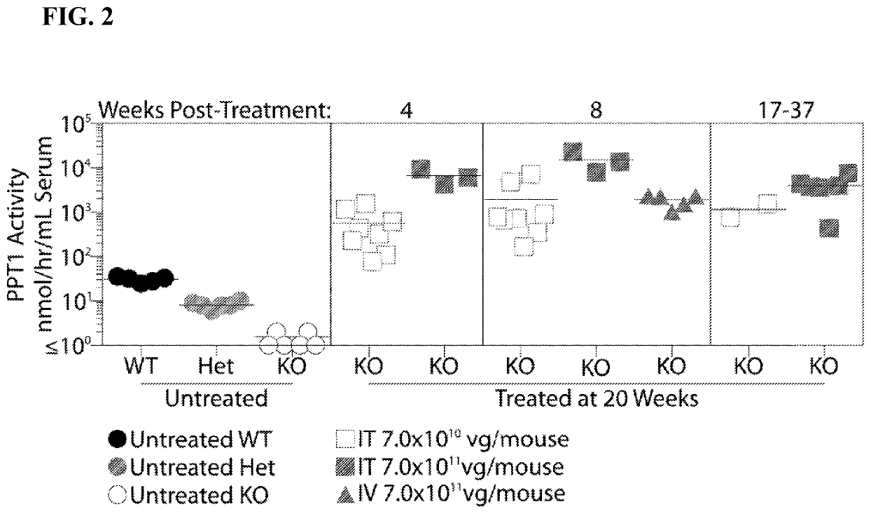

FIG. 2 shows serum enzyme activity of PPT1 in mice administered scAAV9/CLN1 therapy. The vector was injected intrathecally into wild-type, heterologous and CLN1 knockout mice at doses of 7×10¹⁰ or 7×10¹¹ vector genomes, or intravenously at a dose of 7×10″ vector genomes. Vectors were administered at 20 weeks. PPT1 serum enzyme activity was measured at 4 weeks, 8 weeks, and 17-37 weeks post treatment. Supraphysiological PPT1 serum enzyme activity levels were observed at all time points and dosages.

### Example 2

### Combined Intrathecal and Intravenous Administration of AAV Expressing PPT1 Protein Improves the Life Span of CLN1 Knockout Mice

AAV viral particles expressing PPT1 protein were prepared as described in Example 1 and tested for their effect on the life span of CLN1 knockout mice when administered intrathecally and/or intravenously.

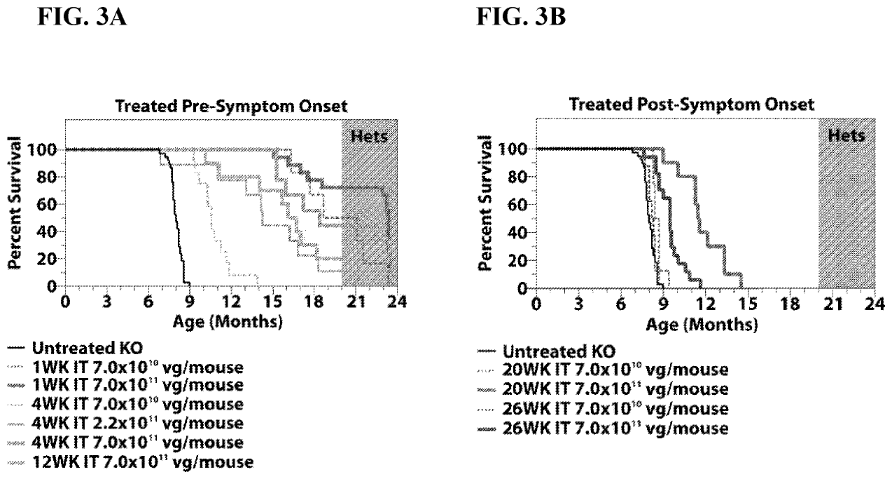

FIGS. 3A-3B show the lifespan of CLN1 knockout mice intrathecal administered with scAAV9/CLN1. Shaded area shows survival range for untreated heterologous mice. In FIG. 3A, various doses of vector genomes were at 1, 4, and 12 weeks, before the onset of symptom. The results showed that intrathecal administration of scAAV9/CLN1 dose-dependently prolongs survival when given at an early age. In FIG. 3B, the vector was injected intrathecally into CLN1 knockout mice at doses of 7×10¹⁰ or 7×10¹¹ vector genomes at 20 or 26 weeks, after the onset of symptom. The results showed that higher dose of scAAV9/CLN1 administered post-symptom onset prolonged survival of CLN1 knockout mice, but the survival benefit was much smaller compared to the survival benefit of scAAV9/CLN1 administration pre-symptom onset.

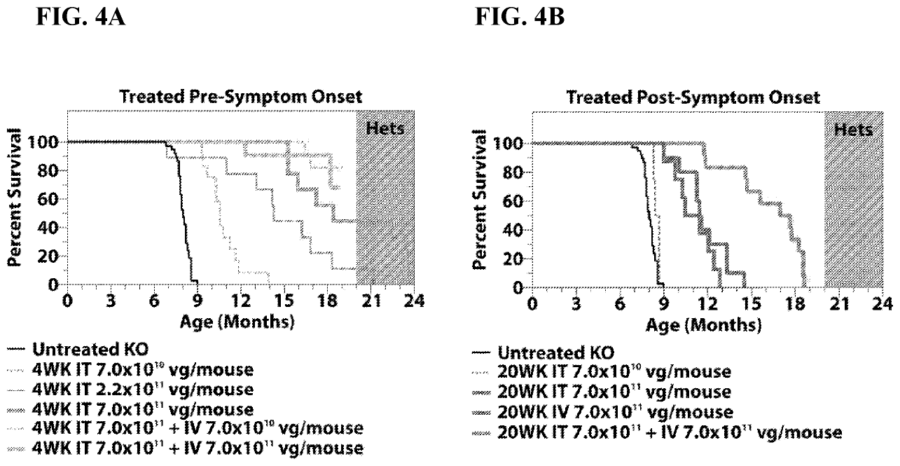

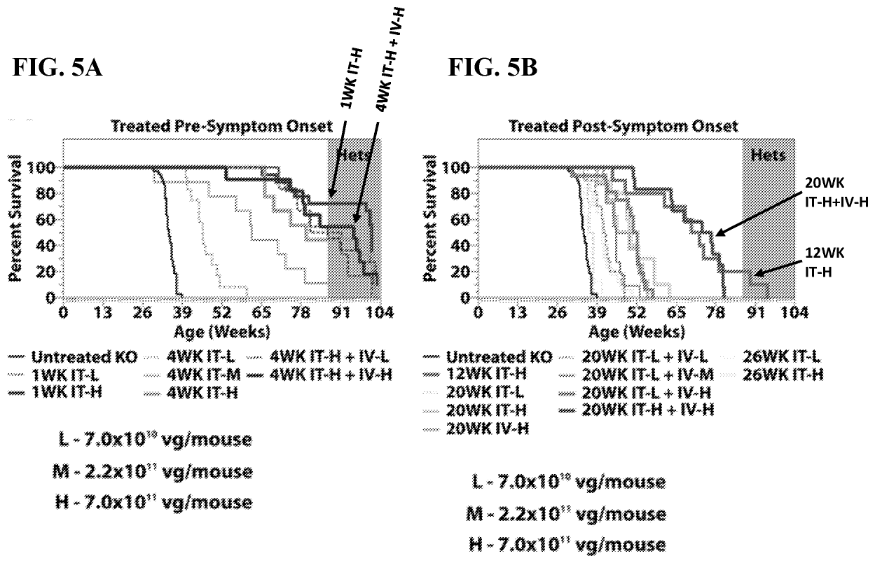

FIGS. 4A-5B show the lifespan of CLN1 knockout mice that received various doses of scAAV9/CLN1 via intrathecal, intravenous, or combined intrathecal and intravenous administration. Shaded area shows survival range for untreated heterologous mice. Notably, FIGS. 4B and 5B show that for CLN1 knockout mice that received scAAV9/CLN1 at 20 weeks (post-symptom onset), the combined intrathecal and intravenous administration offered significantly larger survival benefit as compared to intrathecal or intravenous administration only.

### Example 3

### Combined Intrathecal and Intravenous Administration of AAV Expressing PPT1 Protein Improves the Performance of CLN1 Knockout Mice in Behavioral Assays

Behavioral assays were performed on treated mice to detect improvements in behavior after intrathecal and/or intravenous administration of AAV viral particles expressing PPT1 protein. AAV viral particles were prepared as described in Example 1.

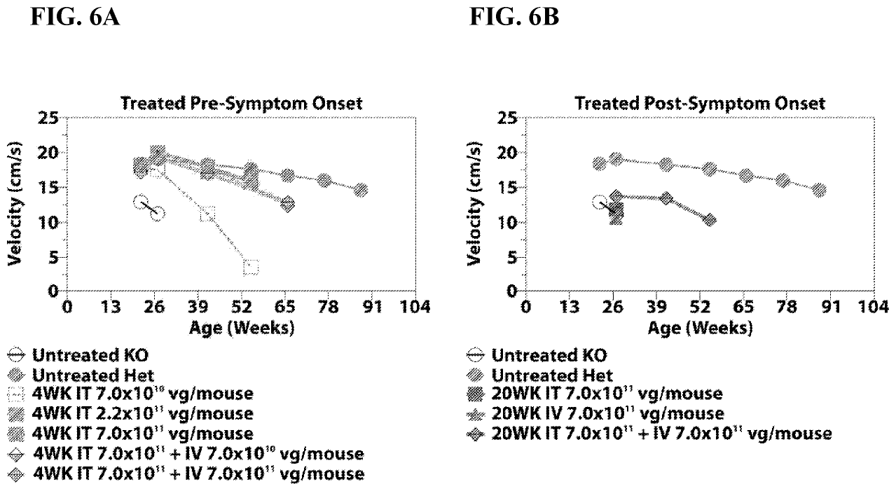

In a test of swimming ability, mice (heterologous untreated, knockout untreated, knockout treated with various doses of vector at 4 or 20 weeks) were placed in a Morris Water Maze consisting of a 122 cm diameter pool filled with 45 cm deep water located in a room with numerous visual cues. Each mouse was given 4 trials per day, across 2-3 days, to swim to an escape platform cued by a patterned cylinder extending above the surface of the water. For each trial, the mouse was placed in the pool at 1 of 4 possible locations (randomly ordered), and then given 60 seconds to find the visible platform. If the mouse found the platform, the trial ended, and the animal was allowed to remain 10 seconds on the platform before the next trial began. If the platform was not found, the mouse was placed on the platform for 10 seconds, and then given the next trial. Tests were carried out at different ages and swim speed was measured. FIGS. 6A-6B show the results. Notably, among knockout mice treated with vector at 20 weeks, those that received the combined intrathecal and intravenous administration of vector displayed significantly slower disease progression, and largely maintained swim speed beyond 52-week age (FIG. 6B), at which point most of the knockout mice that received only intrathecal or only intravenous administration of vector had died.

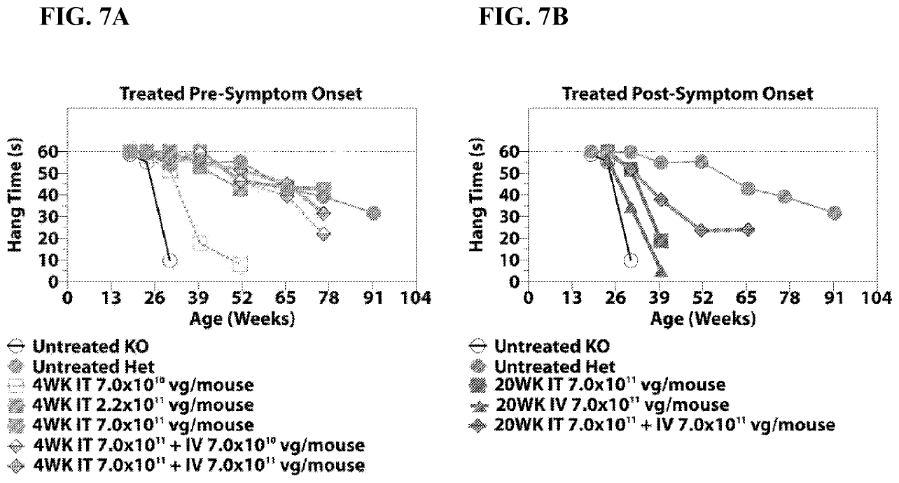

In a test for grip strength, a mouse (heterologous untreated, knockout untreated, knockout treated with various doses of vector at 4 or 20 weeks) was placed on a large metal cage lid. The lid was gently shaken to induce the mouse to grip onto the metal grid. The cage top was then flipped over, and latency for the mouse to fall from the lid was recorded. The maximum trial length was 60 seconds. Tests were carried out at different ages. Time to fall was measured. FIGS. 7A-7B show the results. Notably, among knockout mice treated with vector at 20 weeks, those that received combined intrathecal and intravenous administration of vector displayed significantly slower strength loss and performed better on these tasks than the knockout mice that received only intrathecal or only intravenous administration of vector (FIG. 7B).

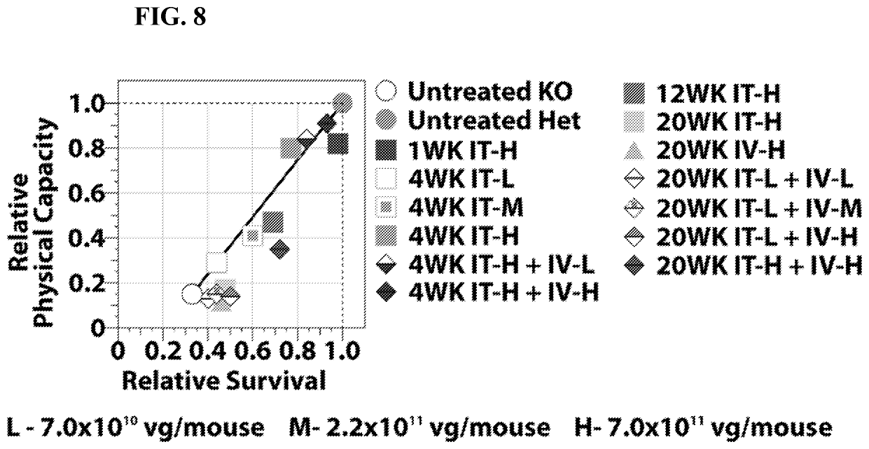

FIG. 8 shows normalized physical capacity score (PSC) vs relative survival time for various mouse treatment groups. PSC was derived from the area under the curve (splines approximation) for the within-treatment averages across time, normalized to heterologous mice at 1. PSC combined data from weight accelerating rotarod, and wirehang. Median survival for heterologous mice was set to 712 days. The results showed a strong correlation between the relative survival time and the relative physical capacity of mice.

### Example 4

Analysis of PPT1 Level and its Physiological Effect in CLN1 Knockout Mice Administered with AAV Viral Particles Expression PPT1 Protein

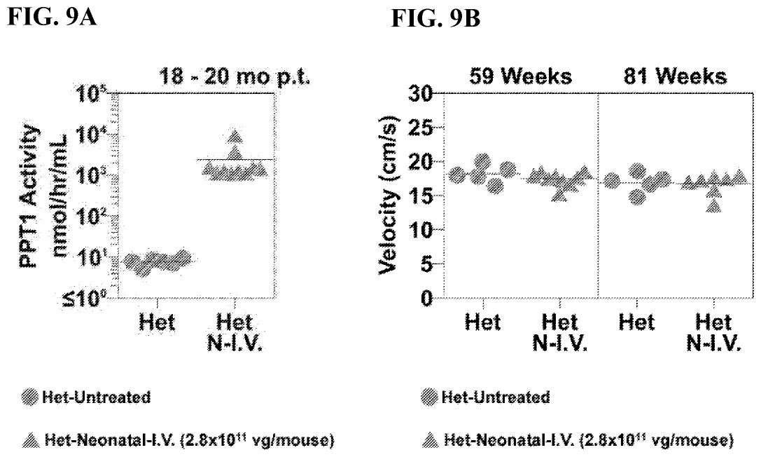

The effect of scAAV9/CLN1 therapy in neonates was tested. Heterologous mice were administered vector (2.8×10¹¹ vg) intravenously as neonates. Serum PPT1 levels (FIG. 9A) and swimming speed (FIG. 9B) were tested at different ages. The results do not show detrimental effects to vector treated heterozygous mice, despite long-term expression of supraphysiological levels of serum PPT1 enzyme activity.

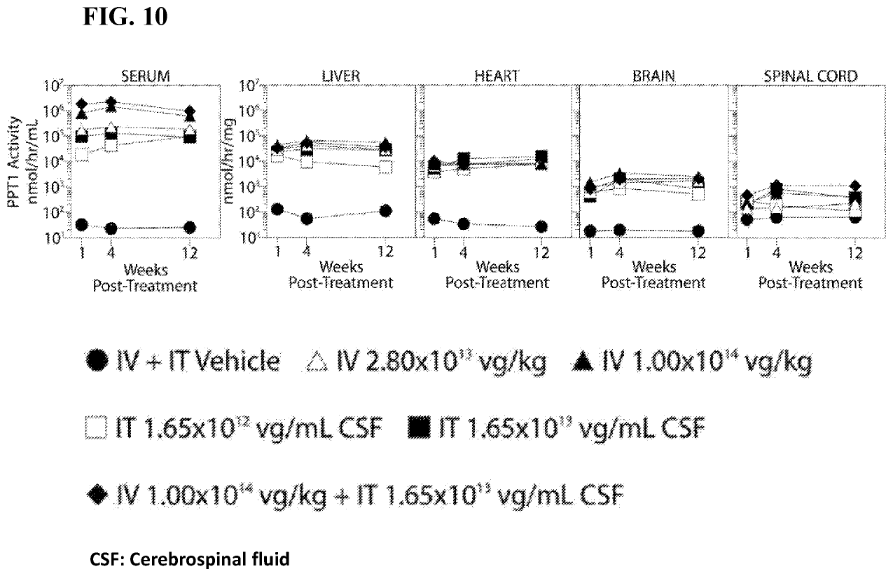

PPT1 enzyme activities were also measured in different tissues of rats treated with scAAV9/CLN1 vector. Rats were treated with either vehicle control or scAAV9/CLN1 vector according to doses and administration routes indicated in FIG. 10. The results show that rats treated with scAAV9/CLN1 vector displayed sustained supraphysiological levels of PPT1 enzyme activity across tissues.

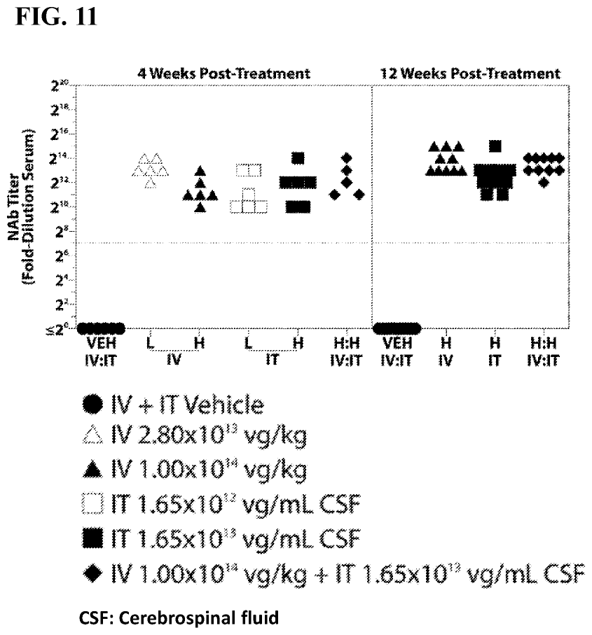

Generation of neutralizing antibody against AAV9 were also tested in rats treated with scAAV9/CLN1 vector. Rats were treated with either vehicle control or scAAV9/CLN1 vector according to doses and administration routes indicated in FIG. 11, and the titer of anti-AAV9 neutralizing antibody were measured at 4 and 12 weeks. The results show that rats developed neutralizing antibody against AAV9 regardless of the administration route of the vector (intravenous, intrathecal, or combined intravenous and intrathecal).

### Example 5

### Combined Intrathecal and Intravenous Administration of AAV Viral Particles Delays Symptom Development and Improves Life Span in CLN1 Knockout Mice

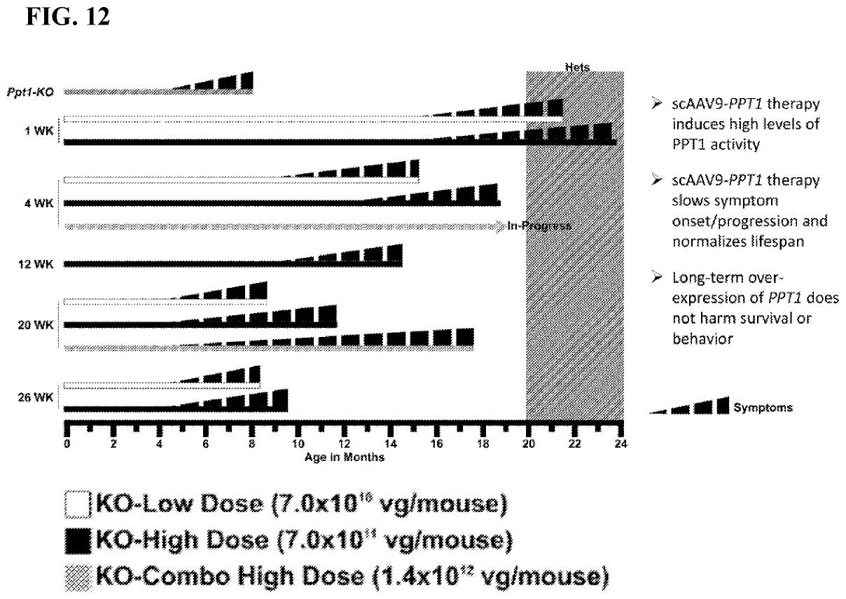

FIG. 12 shows a diagram of symptom development in mice treated with scAAV9/CLN1 vector at different time points (1, 4, 12, 20, and 26 weeks) and via different administration routes (intrathecal or intrathecal+intravenous combo). Shaded area shows survival range for untreated heterologous mice. Overall, early treatment with scAAV9/CLN1 vector provided higher benefit, however, the combined intrathecal and intravenous administration provided significantly higher benefit than intrathecal administration alone when the treatment was administered at a later time point (e.g., 20 weeks).

What is claimed is:

1. A method for treating infantile Batten disease (IBD) or an IBD related disorder in a subject in need thereof, comprising an intrathecal administration of an effective amount of a first AAV viral particle comprising a polynucleotide comprising a CLN1 gene and an intravenous administration of an effective amount of a second AAV viral particle comprising a polynucleotide comprising a CLN1 gene, thereby treating IBD or an IBD related disorder.

2. The method of claim 1, wherein the intrathecal administration precedes the intravenous administration.

3. The method of claim 1 or 2, wherein the polynucleotide of the first AAV viral particle and/or the polynucleotide of the second AAV viral particle comprises a wild-type CLN1 gene sequence, or a codon-optimized CLN1 gene sequence.

4. The method of claim 3, wherein the polynucleotide of the first viral particle and/or the polynucleotide of the second viral particle comprises a codon-optimized CLN1 gene sequence having at least 90% identity to SEQ ID NO: 1, optionally 100% identity to SEQ ID NO:1.

5. The method of any one of claims 1-3, wherein the polynucleotide of the first viral particle and/or the polynucleotide of the second viral particle comprises a nucleotide sequence encoding a polypeptide sequence having at least about 90% identity to SEQ ID NO: 3, optionally 100% identity to SEQ ID NO:3.

6. The method of any one of claims 1-5, wherein the subject is a human patient.

7. The method of any one of claims 1-6, wherein the intrathecal administration and the intravenous administration are performed post-symptom onset.

8. The method of any one of claims 1-6, wherein the intrathecal administration and the intravenous administration are performed pre-symptom onset.

9. The method of any one of claims 1-8, wherein the first and second viral particles are independent selected from an AAV2, an AAV8, an AAV6, an AAV8, and an AAV9 viral particle.

10. The method of claim 9, wherein the AAV viral particle comprises one or more of wild-type capsid proteins, mutated capsid proteins, tissue tropic capsid proteins, or modified capsid proteins having altered tropism compared to a wild-type capsid protein.

11. The method of claim 9, wherein the AAV viral particle is an AAV9 viral particle.

12. The method of any one of claims 1-11, wherein the effective amount for intrathecal administration is from about 1.0×10¹³ vg/kg to about 1.0×10¹⁶ vg/kg, preferably 1.0×10¹⁴ vg/kg to 1.0×10¹⁵ vg/kg.

13. The method of any one of claims 1-12, wherein the effective amount for intravenous administration is from about 1.0×10¹² vg/kg to about 2.0×10¹⁵ vg/kg, preferably 1.0×10¹³ vg/kg to 2.0×10¹⁴ vg/kg.

14. The method of any one of claims 1-13, wherein the polynucleotide of the first viral particle and/or the polynucleotide of the second viral particle is operably linked to a promoter.

15. The method of any one of claims 1-14, wherein the polynucleotide of the first viral particle and/or the polynucleotide of the second viral particle is operably linked to an enhancer.

16. The method of any one of claims 1-15, wherein the polynucleotide of the first viral particle and/or the polynucleotide of the second viral particle is operably linked to an intron.

17. The method of any one of claims 1-16, wherein the polynucleotide of the first viral particle and/or the polynucleotide of the second viral particle is operably linked to a polyadenylation signal.

18. The method of any one of claims 1-17, wherein the first viral particle and/or the second viral particle comprises a vector genome comprising, in 5′ to 3′ orientation, an AAV ITR, an enhancer, a promoter, an intron, a polynucleotide comprising a human CLN1 gene, a polyadenylation site, and an AAV ITR.

19. The method of claim 18, wherein the enhancer is a CMV enhancer, the promoter is a chicken beta actin promoter, the intron is a hybrid/modified MVM intron, and/or the polyadenylation site is a bovine growth hormone polyadenylation site.

20. The method of any one of claims 1-19, wherein the first viral particle and the second viral particle comprise an identical vector genome.

21. The method of any one of claims 1-20, wherein the disorder is infantile, late-infantile, juvenile, or adult-onset neuronal ceroid lipofuscinosis.

22. A kit comprising a pharmaceutical composition comprising the first viral particle and/or the second viral particle in a pharmaceutically acceptable carrier and instructions for use according to the method of any one of claims 1-21.
